(19) United States
(12) Patent Application Publication
(10) Pub. No.: US 2002/0068900 A1
(43) Pub. Date: Jun. 6, 2002

(54) Applicator device, and method for therapeutic massage and dilation of body cavities

(76) Inventors: Robert Scott Barnes, Idaho Springs, CO (US); Shelly Elaine Barnes, Idaho Springs, CO (US)

Correspondence Address: Robert Scott Barnes, 1358 Alps Mountain Road, Box #3052, Idaho Springs, CO, 80452, US

(21) Appl. No.: 09/992,077

(22) Filed: Nov. 6, 2001

Related U.S. Application Data
(60) Provisional application No. 60/245,945, filed on Nov. 6, 2000.

(51) Int. Cl. A61M 29/00

(52) U.S. Cl. 604/107; 604/108

(57) ABSTRACT

A Device and Method for providing Therapeutic Massage and Dilation is disclosed. The Device is a uniquely designed instrument that is cupped from side to side and is narrower at one end than at the other. The narrow end has a flattened tip that is rounded around its perimeter. The wide end incorporates a semi-spherical handle. The bottom of the Device is convex and has small ridge that is rounded running along most of its length.

The primary function of the present invention is for use in the vagina to loosen and stretch the perineum thereby reducing trauma at birth. Depending on the size of manufacture, body cavities other than the vagina can benefit from internal massage for the relief of deformity, stricture, or other discomforting conditions.

The instrument is used by inserting it into the vagina with its bottom side to the vaginal floor. Then the device is moved from side to side in a rocking motion to massage the surrounding tissues. The flattened tip can be used to pull the perineal tissues forward as they begin to loosen, the flattened tip can be used to massage tissues that are tight as well. The cupped or concave surface on the top side of the instrument helps prevent contact with cervix and urethra thereby avoiding irritation to these parts of the female anatomy, it also provides a recessed surface for the attachment of an optional reservoir for the application of ointments or medications. The handle is dual purpose and can be used to massage the tissues at the opening of the vagina. A provision for the attachment of a vibration generating device is also included thereby offering additional therapeutic value to certain users.

### RELATED APPLICATIONS

This is a continuation-in-part of pending U.S. Provisional Patent Application Ser. No. 60/245,925 filed Nov. 6,2000, the contents of which is specifically incorporated herein by reference.

There are also references made to the following U.S Patents for the purpose of illustrating prior art that has been designed for the purpose of producing similar results as the device claimed herein.

U.S. Pat. No. 6,224,580 May 01,2001 Christenson, ENTITLED Apparatus for treatment of the female pelvic floor.

U.S. Pat. No. 3,799,170 Mar 26,1974 Walsh et al., ENTITLED Therapeutic Dilator, Disposable or Reusable.

U.S. Pat. No. 2,026,747 Jan 07,1936 Nemzek, ENTITLED Gravity Thermal Dilator.

U.S. Pat. No. 899,477 Sep 22,1908 Williams, ENTITLED Dilator and Applicator.

### FEDERALLY SPONSORED RESEARCH OR DEVELOPMENT

Not Applicable

### REFERENCE TO MICROFICHE APPENDIX

Not Applicable

### BACKGROUND OF THE INVENTION

1. Field of the Invention

The present Invention relates generally to instruments and devices used for dilation of body cavities, specifically the present invention relates to devices engineered for the purpose of stretching vaginal or perineal tissues and or muscles.

2. Description of Prior Art

Current Instruments and Devices that are used for dilation and stretching of tissues and muscles of body cavities generally consist of an inflatable apparatus designed for insertion into a body cavity and when properly positioned it is the then inflated to an appropriate size that then provides the desired stretching force on the surrounding body tissues. Examples of this type of device are found in U.S. Pat. No. 6,224,580 and in U.S. Pat. No. 3,799,170. Some Instruments used for dilation of body cavities. incorporate a therapeutic feature such as a provision for heating the device and/or a provision for administering medicine or an ointment during the use of the device. Examples of these types of devices can be found in U.S. Pat. No. 2,026,747 and in U.S. Pat. No. 899,477.

The devices referred to were developed due to a need to dilate various body cavities for the purpose of treating a variety of conditions, including deformity, strictures, sexual dysfunction/discomfort, and more recently to reduce trauma to vaginal tissues at birth.

The current devices referred to can be somewhat complicated and may require the assistance of an individual familiar with, or especially trained in the use of the device for safe and effective operation thereof. Furthermore some of the current devices may require regular maintenance to function both safely and properly.

Because there is currently a greater focus on female health issues including that of perineal care there is a need for a device that;

1. Does not require a trained professional to operate,

2. Can be used in the privacy of the home,

3. Requires little or no maintenance,

4. And is inexpensive to produce and distribute.

The previously referred to U.S. Provisional Patent No. 60/245,925 fills these needs and provides additional functional and beneficial features utilizing a massage technique that can not possibly be used properly by previously designed instruments for dilation of body cavities. A combination of optional features as disclosed herein further enhance the performance of the invention.

### SUMMARY OF THE INVENTION

Accordingly it is one object of the present invention to provide a Device designed for the purpose of providing therapeutic massage to the interior of a body cavity, more particularly to, but not limited to the vagina.

It is another object of the present invention to provide a Device designed to dilate a body cavity though both massage and stretching force, more particularly to, but not limited to the vagina.

Yet another object of the invention is to provide a Device for the administration of ointments or medications while at the same time providing therapeutic massage to the internal surface of a body cavity, more particularly to, but not limited to the vagina.

Still it is another object to provide such a Device as stated with a low cost of manufacture, such as injection molding of non-porous materials, thermoforming of non-porous materials, or possible casting of non-porous materials. While plastic construction of the Device is the preferred method, other methods and materials of manufacture, including polishing or finishing of porous materials may be suitable.

To achieve the forgoing objects and other objects and in accordance with the purpose of the present invention, as embodied and broadly described herein, an Applicator Device For Therapeutic Massage and Dilation of Body Cavities is disclosed. The design of the Device is such that it comprises of an instrument formed in a tapered shape that includes a small ridge along its bottom side, a concave surface along its top side, a flattened tip that is rounded from side to side and around its edges at the narrow end, and an integral handle at its wide end that is convex on its outer surface as it curves upward from the bottom side of the instrument. Other styles of handles that vary from the present invention may be used depending on the need of the user. The Device may be manufactured in a variety of sizes in order to fit the need of the user.

The device is used by inserting the tapered end into a body cavity such as the vagina, and while applying pressure, rocking the device from side to side stretching the tissues across the bottom of the instrument and over the small ridge thereby effectively dilating and loosening the surrounding tissues. Various amounts of dilation can be achieved with more aggressive massage and additional penetration. The flattened tip of the device can be used to apply direct pressure to the interior walls of the body cavity for the purpose of shaping the tissues through massage. When the device is equipped with the integral handle described above, the handle itself can be used to massage and stretch the tissues at the opening of the body cavity, this would include the perineal tissues in a female user. The device also includes a provision for mounting a vibration generating device that further enhances the therapeutic value for some users.

The Device herein disclosed may have numerous uses in a variety of body cavities, but is particularly valuable when used in the vagina due to its unique design. The concave surface along the top prevents cervical and urethral irritation during use, furthermore the small rounded ridge on the bottom is also unique in that it provides a more direct contact point for applying pressure during massage. The broad semi-spherical handle as described is unique in that it provides an ergonomic surface for controlling the use of the device as well as providing a larger surface for additional massage at the opening of the body cavity. There is also a provision for use of an optional disposable or reusable reservoir that can be attached via an adhesive to the concave top surface of the invention for the purpose of administering ointment or medication during the use of the device.

### BRIEF DESCRIPTION OF THE DRAWINGS

The accompanying drawings are provided as part of the specification to illustrate the preferred embodiments of the present invention, and together with the description, serve to explain the nature and function of the invention. In the drawings:

FIG. 1 is a side perspective view of the present invention, including a typical vibration generating device that may be used in conjunction with the present invention.

FIG. 2 is a cross-sectional view from the side, providing an illustration of the profile of the structure of the present invention.

FIG. 3 is a cross-sectional view through the body of the present invention, providing a typical view of the structure from the tip of the device to the rear handle feature.

FIG. 4 is a plan view from the bottom showing the overall shape of the device, it also shows the location of the rounded ridge portion of the present invention that is not clearly shown in FIG. 1

FIG. 5 is a perspective view of the optional reservoir in the same orientation as FIG. 1

### DETAILED DESCRIPTION OF THE DRAWINGS

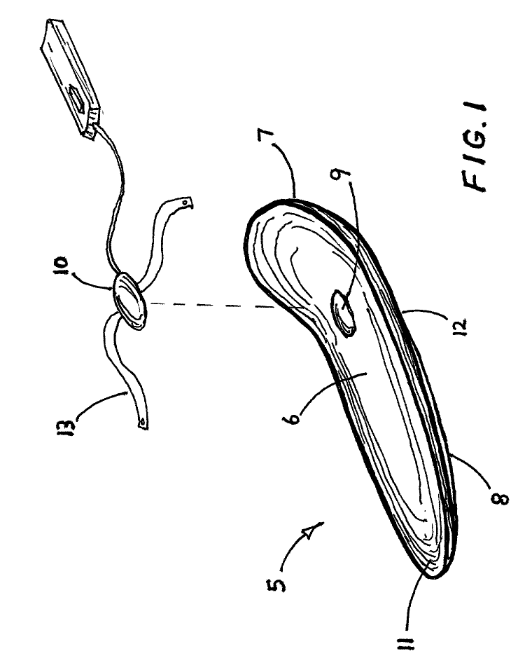

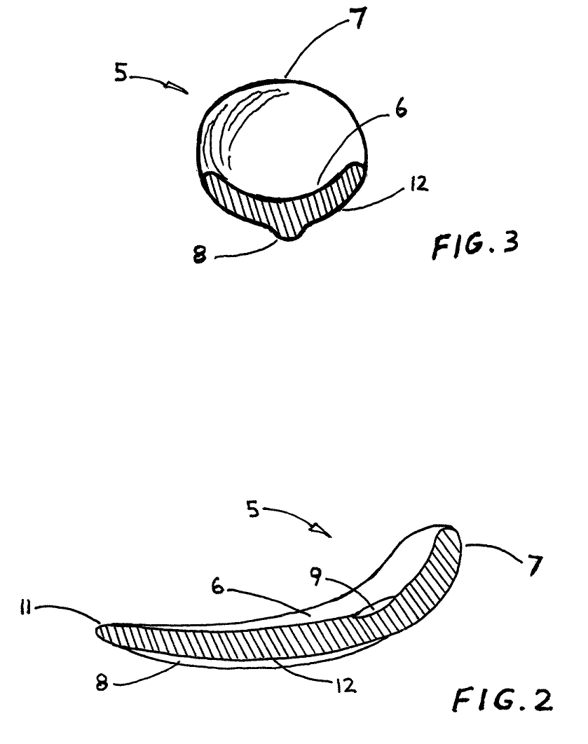

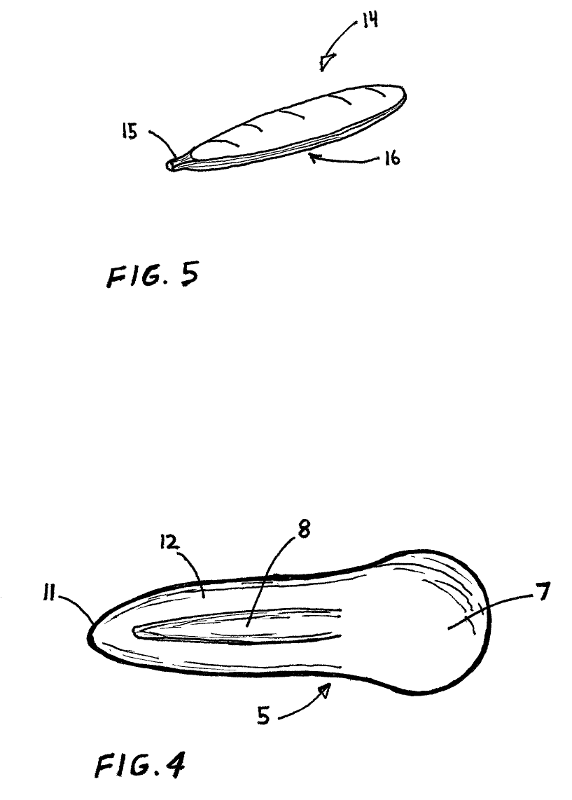

Referring to FIG. 1, FIG. 2, FIG. 3, and FIG. 4 a Device 5 for dilation and massage of body cavities is shown.

Referring to FIG. 5 an optional reservoir 14 is depicted.

FIG. 1 specifically shows an overall view of the Device 5 including a representation of a type of vibration generating device 10 that may be suitable for attachment to the Device 5 with a strap 13, furthermore FIG. 1 generally illustrates the cupped or concave nature of the upper surface 6, the flattened and rounded tip 11, the semi-spherical handle 7, the depression 9 for use as a mounting point of a vibration generating device 10, and a portion of the rounded ridge 8 that is located on the lower or bottom surface 12 of the Device 5.

FIG. 2 specifically shows a cross-sectional view of the Device 5 in its length, further illustrating the shape of the semi-spherical handle 7 of the present invention, the ridge 8 on the bottom surface 12, the rounded tip 11, and the mounting depression 9.

FIG. 3 specifically shows a cross sectional view though the middle of the Device 5 looking from the front to the rear. This view illustrates the general shape from side to side of the Device, allowing for the recessed attachment of the optional reservoir 14 depicted in FIG. 5, as well as the general shape of the ridge 8 on the bottom surface 12. The general shape of the upper surface 6, and the inner face of the semi-spherical handle 7 at the rear can be seen as well.

FIG. 4 specifically shows a plan view of the Device 5 from the bottom and illustrates the placement of the ridge 8 on the bottom surface 12. Orientation of the rounded tip 11, and semi-spherical handle 7 is also depicted.

FIG. 5 illustrates a disposable or reusable bag type reservoir 14 in perspective, and integral spout or nozzle 15 for the storage and administration of ointments during the use of the Device 5, when attached to the inner or top surface 6 as depicted in FIG. 1, and FIG. 3 with a form of adhesive 16.

### DETAILED DESCRIPTION OF THE INVENTION

This invention generally relates to dilators, particularly to therapeutic dilators, more particularly to those designed to treat conditions in the vagina. This invention may however be used to treat conditions in other body cavities such as various types of stricture caused by disease, injury, deformity, and other causes.

The primary purpose of this invention is to treat vagina strictures or deformity, and more commonly to treat or prepare the vaginal floor and perineum in a pregnant woman during the final trimester of pregnancy in order to reduce trauma at birth.

The invention consists of a device that is especially designed with a tapered shape for penetration of a body cavity such as the vagina. The narrow end is inserted and the device pushes the tissues outward as with a traditional dilator. Depending on the depth of penetration the device due to its shape will put a varying amount of pressure on the walls of the body cavity, however this not the only nor is it the primary force used for therapeutic dilation with this device.

The device described herein has special features that allow it to be used as a massage instrument, thereby providing therapeutic dilation via a massage technique rather than dilation via pure stretching force from expansion.

The body of the stated device is similar in shape to a cone that has been cut in half through the middle from the point to the base, with all the edges rounded off. This cone shape is then hallowed out forming a cavity or recess on its flat side. The tip is slightly flattened, the base is expanded and turned upward forming semi-spherical shape that can be used as handle. The round side is considered the bottom and has small ridge that is formed into it in a rounded manner for most of the length if the device. This unique shape is accomplished in manufacture by means of injection molding, thermoforming, casting, or other methods of shaping. The preferred method of manufacture is that of injection molding of plastic. This method of injecting heated plastic under pressure into a polished mold allows for the use of a non-porous and heat resistant plastic, of which several types are readily available and F.D.A. approved. This type of material allows for a device that can be properly sanitized between uses, and allows for the device to be heated or warmed prior to use, thereby enhancing its therapeutic value and comfort.

After the device has been inserted into a body cavity such as the vagina, pressure is applied in a downward direction and the device is rocked from side to side causing the tissues in the body cavity to be stretched across the rounded ridge along the bottom side of the device. This unique feature allows the tissues to be stretched or dilated more comfortably. Another important feature is the convex shape of the top side of the device, in that because the device is not round, contact with certain tissues of the body cavity can be minimized or avoided. This is particularly important when used on the perineal tissues in the vaginal area because aggressive massage and dilation in this area can cause irritation or soreness to the urethra and other tissues near the pelvic bone.

The present invention also includes a rounded tip that has been slightly flattened so that it may be used as a stylus during the massage routine. The tip can be used to apply a focused amount of pressure to a particular part of the body cavity or used in a raking motion to shape or pull forward the tissues of the body cavity. The raking motion as described has been found to be effective in producing a bowel movement in women when used on the pelvic floor.

The ergonomic handle of the invention is ingenious in that it has a dual purpose. Because of its unique semi-spherical shape it can be used to control the use of the device effectively, as well as be used as a massage surface. When the handle is used as massage surface the device is reversed and the convex side of the handle is then used to apply pressure directly to the tissues at the opening of the body cavity, to conduct the massage the device is rolled about the opening of the body cavity in a manner similar to that used by a mortise and pestle.

Other features that are optional to the use of the devise are the use of a vibration generating device, or the use of a removable reservoir for the application of ointments or medications. The vibration generating device is not herein claimed and there may be several types suitable for use, however a provision for mounting such a device is included in the form of an additional depression in the upper concave surface of the invention. The use of a removable reservoir is also facilitated in that it can be mounted within the concave surface between the tip and handle of the device with the discharge nozzle or opening oriented towards the tip. The reservoir is constructed of flexible material, such as some types of plastics and rubbers, and is mounted with a temporary type of adhesive. The ointment or medication is dispensed by applying pressure to the reservoir causing it to be released into the body cavity. The reservoir may be disposable or reusable. The device may be used as reservoir itself without the removable reservoir by placing an ointment or medication in the concave top side of the device where the removable reservoir would normally be affixed. This would allow for substantially larger dose of ointment or medication to be applied to the interior of the body cavity than a simple application of ointment or medication to the exterior of a traditional dilator.

While the device is particularly suitable for use in the vagina of human females, the present invention can be used to treat conditions in other body cavities such as the rectum, or to treat conditions in the body cavities of other animals as well, depending upon the size of manufacture and other modifications that do not change the intended purpose of dilating a body cavity though therapeutic massage.

1. We claim an applicator device for the purpose of performing therapeutic massage and dilation of body cavities with specific features that enhance the use of said device in the vagina.

2. We claim the device as claimed in claim #1 wherein said device has a cupped profile in cross-section allowing said device to be used in a body cavity in such a manner as to not effect a particular portion of the body cavity.

3. We claim the device as claimed in claim #1 wherein said device has a rounded ridge along its lower convex surface providing said device with a pressure point for use during internal massage.

4. We claim the device as claimed in claim #1 wherein said device is tapered in its length and raised at its edges allowing for a varied amount of dilation during massage as well as providing a form that is in effect a reservoir for holding ointments or medications for the purpose of internal application of such into a body cavity, or for the recessed placement of an optional removable reservoir, disposable or reusable, containing ointments or medication for the application thereof into a body cavity.

5. We claim the device as claimed in claim #1 wherein said device has a flattened tip at its narrow end allowing the device to be used for applying direct pressure on a specific internal portion of a body cavity.

6. We claim the device as claimed in claim #1 wherein said device is constructed of a non-porous, heat resistant plastic, that it may retain its original shape when warmed for use or for cleaning.

7. We claim the device as claimed in claim #1 wherein said device incorporates a dual purpose handle that is semi-spherical in its shape providing both an ergonomic handle for controlling the device and providing an additional massage surface for the device.

8. We claim the device as claimed in claim #1 wherein the body of said device is constructed as one seamless piece with the handle portion as clamed in claim #7.

9. We claim the device as claimed in claim #1 wherein said device contains an optional depressed mounting provision for a vibration generating device.

10. We claim the optional bag type reservoir, disposable or reusable when configured for recessed attachment and use with the device as claimed in claim #1.

11. We claim the device as claimed in claim #1 wherein said device has additional applications other than that of vaginal massage, depending upon size of manufacture, both human and veterinary.
